(19) United States
(12) Patent Application Publication
(10) Pub. No.: US 2016/0045306 A1
(43) Pub. Date: Feb. 18, 2016

(54) CUT PATTERN TRANSCATHETER VALVE FRAME

(71) Applicant: BOSTON SCIENTIFIC SCIMED, INC., Maple Grove, MN (US)

(72) Inventors: Sumit Agrawal, Woodbury, MN (US); Ismail Guler, Maple Grove, MN (US); David M. Flynn, Lino Lakes, MN (US); Paul F. Chouinard, Maple Grove, MN (US); Kenneth M. Merdan, Loretto, MN (US)

(73) Assignee: BOSTON SCIENTIFIC SCIMED, INC., Maple Grove, MN (US)

(21) Appl. No.: 14/823,691

(22) Filed: Aug. 11, 2015

Related U.S. Application Data
(60) Provisional application No. 62/053,421, filed on Sep. 22, 2014.
(60) Provisional application No. 62/038,728, filed on Aug. 18, 2014.

(51) Int. Cl. A61F 2/24

(52) U.S. Cl. CPC A61F 2/2409; A61F 2/243; A61F 2210/0014; A61F 2210/0057; A61F 2220/0033; A61F 2220/0041; A61F 2230/0069; A61F 2230/0017; A61F 2230/0004; A61F 2230/0058; A61F 2230/0021; A61F 2230/0056; A61F 2240/001

(57) ABSTRACT

A transcatheter valve frame comprising a valve frame comprising an elongate tubular member having a longitudinal axis and a circumference, the elongate tubular member comprising a plurality of struts interconnected by peaks and valleys that define a closed cell construction comprising a plurality of interconnected cells, the valve frame is formed from a single piece of material comprising a metal or metal alloy, the valve frame having a diameter that increases upon axial compression and the valve frame comprising a locking mechanism integral with the valve frame configured to fix the diameter of the valve frame.

### CROSS-REFERENCE TO RELATED APPLICATIONS

This application claims the benefit of and priority to U.S. Provisional Application No. 62/053421, filed Sep. 22, 2014, and this application claims the benefit of and priority to U.S. Provisional Application No. 62/038,728, filed Aug. 18, 2014, the entire contents of which are herein incorporated by reference.

### BACKGROUND OF THE INVENTION

The present invention relates to methods and apparatus for endovascularly repairing or replacing a heart valve.

Valvular heart disease (VHD) is any disease that affects one or more valves of the heart and often requires elaborate diagnostic procedures, intervention, and long-term management. Heart valve replacement traditionally involved open heart surgery with associated risks including, high mortality, incidence of neurological damage, stroke, and repeated valve replacement.

Minimally invasive procedures have now been developed for the valve replacement that are much less traumatic and reduce the risks associated with surgical valve replacement.

Percutaneous valve implantation including transcatheter aortic valve implantation (TAVI), percutaneous aortal valve implantation (PAVI) and percutaneous pulmonary valve implantation (PPVI) are less invasive alternatives to open heart surgery for patients in need of heart valve replacement. In percutaneous valve implantation, prosthetic implants are delivered through catheters using transvenous, transarterial, or transapical techniques.

Replacement heart valves generally include a valve frame and valve leaflets disposed within and attached to the valve frame. Some conventional valve frames are formed of a braided construction comprising a metal or metal alloy. The braiding process is time consuming and can result in manufacturing inefficiency. Moreover, a braided frame may exhibit fretting wear caused by relative motion of one strand of the braid that is in contact with another strand of the braid. Such wear can decrease the expected useful lifespan of the device.

Without limiting the scope of the invention, a brief summary of some of the claimed embodiments of the invention is set forth below. Additional details of the summarized embodiments of the invention and/or additional embodiments of the invention may be found in the Detailed Description of the Invention below.

### SUMMARY OF THE INVENTION

In some aspects, the present invention relates to a transcatheter valve frame comprising a valve frame comprising an elongate tubular member having a longitudinal axis and a circumference, the elongate tubular member comprising a plurality of struts interconnected by peaks and valleys that define a closed cell construction comprising a plurality of interconnected cells, the valve frame is formed from a single piece of material comprising a metal or metal alloy, the valve frame having a diameter that increases upon axial compression and the valve frame comprising a locking mechanism integral with the valve frame configured to fix the diameter of the valve frame.

The transcatheter valve frame may have a plurality of cells that define a uniform pattern throughout the elongate tubular member.

The transcatheter valve frame may have a plurality of cells that define a uniform pattern throughout the elongated tubular member wherein the plurality of cells comprise arcuate-shaped diagonal elements.

The transcatheter valve frame may comprise a plurality of cylindrical bands, within each cylindrical band each of the arcuate-shaped diagonal elements is oriented in the same direction about the circumference of the elongate tubular member.

The transcatheter valve frame may have arcuate-shaped diagonal elements that have a less arcuate shape in a stretched condition than in a non-stretched condition such that the diagonal elements at least partially straighten for minimizing foreshortening of the elongate tubular member along the longitudinal axis of the elongate tubular member.

The transcatheter valve frame may have a plurality of struts connected by peaks and valleys that are arranged in a plurality of cylindrical bands formed in the elongate tubular member, each cylindrical band forming a generally zig-zag pattern comprising that forms a series of sequential diagonal elements connected to one another and extending about the circumference of the elongate tubular member.

The transcatheter valve frame may have a plurality of longitudinal connectors extending between and connecting adjacent bands.

The transcatheter valve frame may have each of the sequential diagonal elements comprising first and second generally straight portions having first and second ends, the second ends being connected together by a curved portion, the first ends being connected to preceding and succeeding diagonal elements.

The transcatheter valve frame may have an elongate tubular member that is radially expandable between an unexpanded state and an expanded state.

The transcatheter valve frame may have a plurality of cells that are honeycomb-shaped.

The transcatheter valve frame may have a plurality of cells that are diamond-shaped.

The transcatheter valve frame may have a plurality of cells that are generally bat-shaped wherein each cell defines a head region, a tail region and opposing curved wing regions, and a plurality of connectors extending between and connecting adjacent cells.

The transcatheter valve frame may have an elongate tubular body having a plurality of cylindrical bands, each cylindrical band comprising a sequence of the generally bat-shaped cells, adjacent cylindrical bands are connected to one another by longitudinal connectors.

The transcatheter valve frame may have a plurality of cells having a four-lobed shape wherein each lobe is uniformly spaced from an adjacent lobe.

The transcatheter valve frame may have a locking mechanism that is a buckle or post integral with the valve frame.

The transcatheter valve frame may have an elongate tubular member formed from a shape memory alloy.

The transcatheter valve frame may be formed from nitinol.

In another aspect, the present invention relates to a method of making a valve frame, the method comprising providing a elongate tubular member formed from a continuous piece of material and cutting a pattern into the elongate tubular member, the pattern comprising a plurality of struts interconnected by peaks and valleys that define a closed cell construction comprising a plurality of interconnected cells to form the valve frame.

The method may further comprise forming a locking mechanism in the valve frame.

The method may further comprise forming the locking mechanism in the valve frame by laser cutting or micromachining

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1A is partial view of one embodiment of a valve frame according to the invention in an expanded state.

FIG. 1B is a partial view of valve frame similar to that shown in FIG. 1A in a crimped state.

FIG. 1C is a partial perspective view of a valve frame similar to that shown in FIGS. 1A and 1B in an expanded state.

FIG. 1D is partial views of a single cell of a valve frame according to the invention.

FIG. 1E is a partial view of a valve frame similar to that shown in FIG. 1D illustrating the effect on the cell of an applied load simulating that which would be applied during a crimping process.

FIG. 1F is a partial view of a single cell similar to that shown in FIG. 1D illustrating the effect on the single cell when compressed axially as during expansion.

FIG. 2 is a partial flat view of another embodiment of a valve frame according to the invention

FIG. 3 is a partial side view of another embodiment of a valve frame according to the invention.

FIG. 4A is a partial flat view of another embodiment of a valve frame according to the invention.

FIG. 4B is a partial expanded view the valve frame taken from section 4B in FIG. 4A.

FIG. 4C is a partial flat view of a valve frame similar to that shown in FIGS. 4A and 4B in a compressed state.

FIG. 4D is a single cell of a valve frame similar to that shown in FIGS. 4A-4C.

FIG. 4E is a single cell of a valve frame similar to that shown in FIG. 4D in a compressed state.

FIG. 4F is a single cell of a valve frame similar to that shown in FIG. 4D illustrating the effect of a load exerting axial compression on the cell as during a valve frame expansion.

FIG. 5 is a partial perspective view of a valve frame having an integral locking mechanism.

FIG. 5A is a partial enlarged view of an integral locking mechanism taken at section 5A in FIG. 5.

FIG. 6 is a cross-sectional view of a valve frame having valve leaflets coupled thereto.

### DETAILED DESCRIPTION OF THE INVENTION

While embodiments of the present disclosure may take many forms, there are described in detail herein specific embodiments of the present disclosure. This description is an exemplification of the principles of the present disclosure and is not intended to limit the disclosure to the particular embodiments illustrated.

The following detailed description should be read with reference to the drawings in which similar elements in different drawings are numbered the same. The drawings, which are not necessarily to scale, depict illustrative embodiments and are not intended to limit the scope of the invention. Those skilled in the art will recognize that the dimensions and materials discussed herein are merely exemplary and are not intended to limit the scope of the present invention.

The present invention relates generally to a replacement heart valve wherein the frame of the heart valve is formed from a cut pattern elongate tubular member formed of a metal or a metal alloy. The frame has the characteristics of elongating when its diameter is reduced and the diameter increases when its length is decreased. The cut pattern frame eliminates fretting wear that can occur with a conventional braided frame because with a cut pattern frame there no strands that are movable with respect to an adjacent strand as in a braided frame.

The pattern formed in the elongate tubular member is suitably a closed cell construction in which a plurality of struts are interconnected by peaks and valleys. Connectors may extend between adjacent circumferential bands of cells, typically oriented in the same direction. This is explained in more detail below with respect to the following figures.

The closed cell-construction may have a plurality of cells comprising arcuate-shaped diagonal elements. This does not preclude, however, the plurality of cells having straight diagonal elements as well.

In some embodiments, the pattern is cut using a laser. However, other fabrications including EDM (electrical discharge machining), ECM (electrochemical machining) or PEM (precise electrochemical machining) may be employed as well.

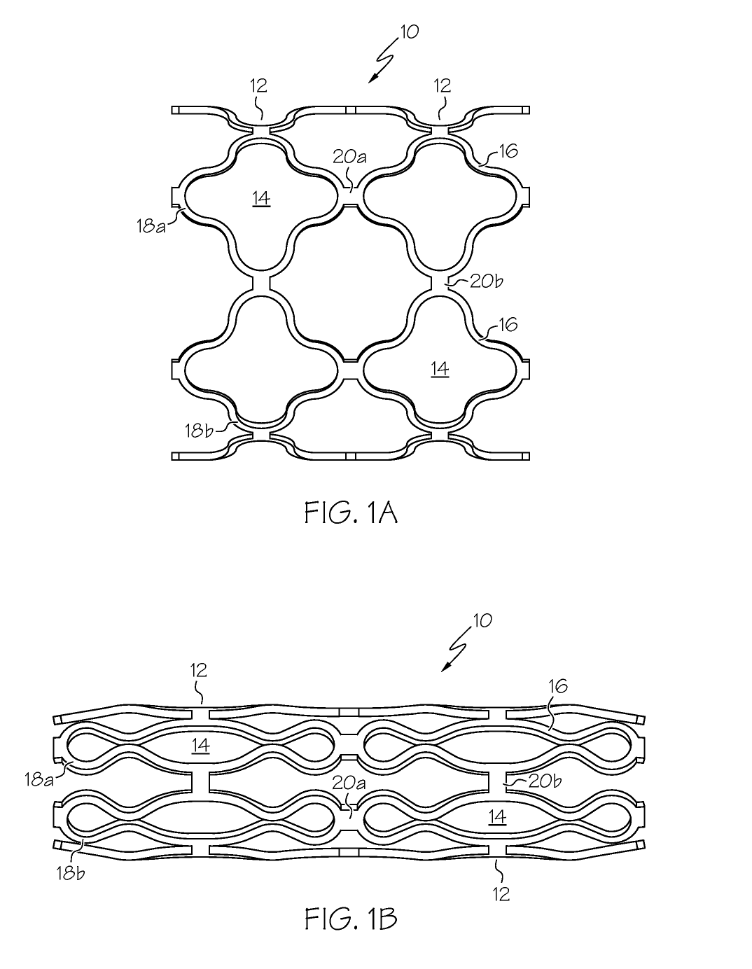

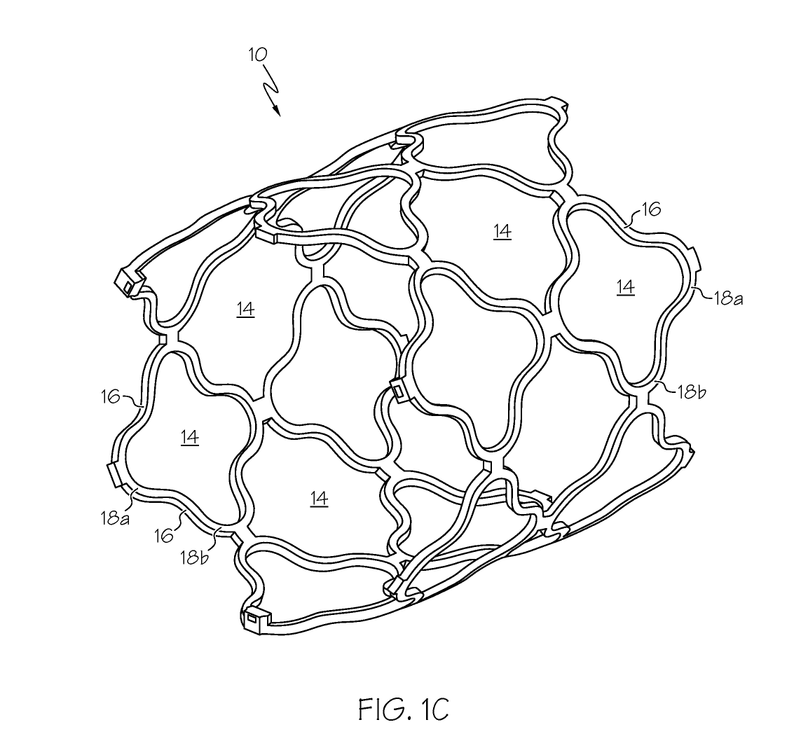

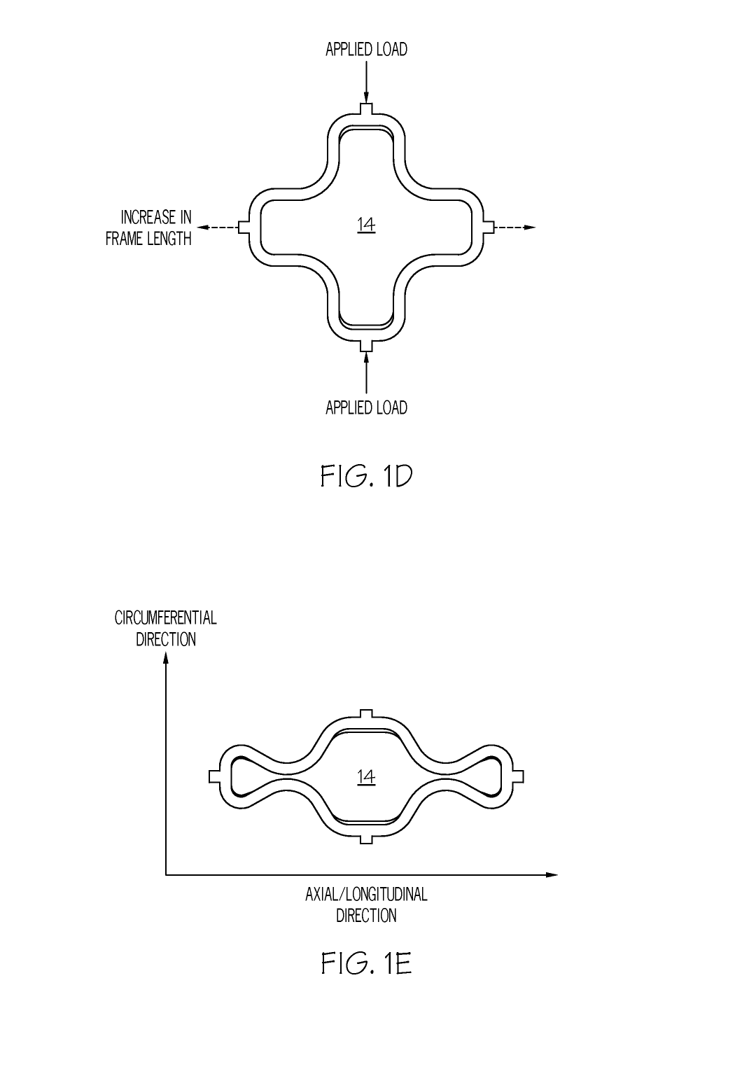

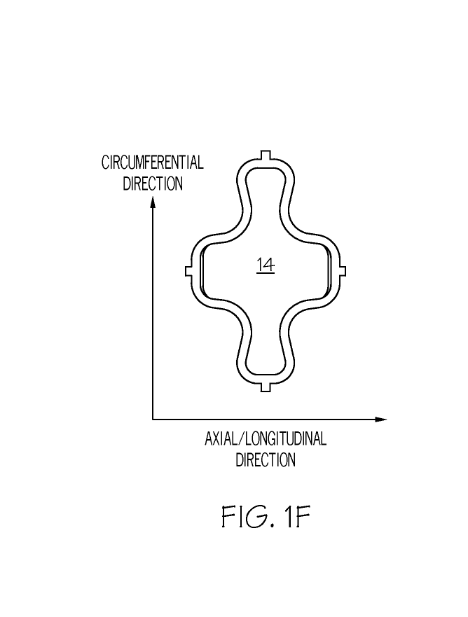

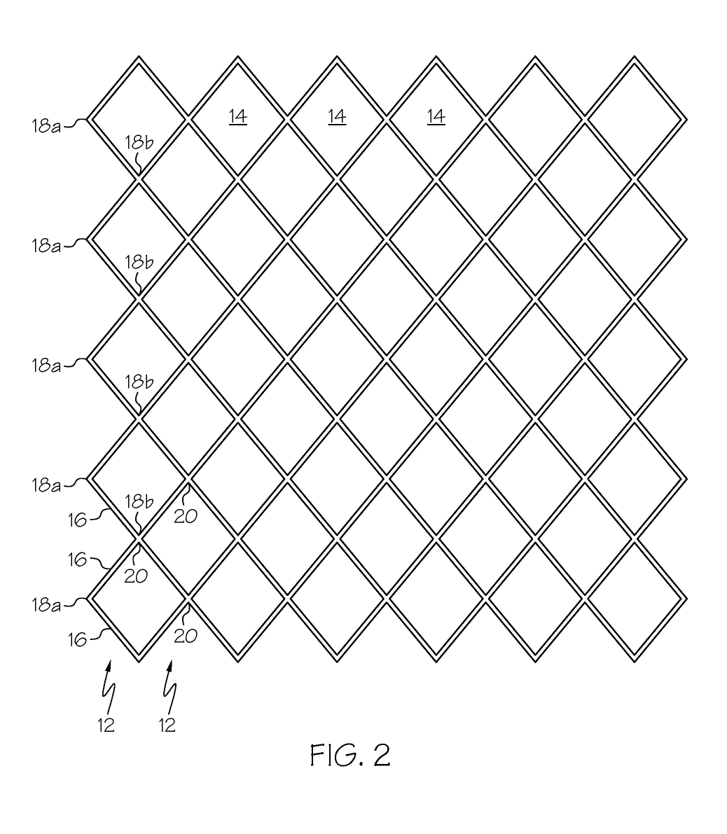

Turning now to the figures, FIG. 1A is partial view of one embodiment of a valve frame 10 in an expanded state having cylindrical bands 12 comprising a plurality of a plurality of cells 14. The cells are formed of struts 16 interconnected by peaks 18a and valleys 18b. In this embodiment, each cell 14 is in the form of a four-lobed shape wherein each lobe is either a peak 18a or a valley 18b. The each cylindrical band 12 is interconnected to an adjacent cylindrical band 12 by longitudinal connectors 20a while connectors 20b perpendicular to the longitudinal axis of the valve frame 10 connect adjacent cells 14.

As used herein, a “peak” is where two struts come together and a “valley” is where the struts come together on the opposing side of a peak. “Connector” is where adjacent cylindrical bands are interconnected.

The four lobes of each closed cell of the valve frame can be formed from a different size for achieving different axial or radial compression.

The valve frame 10 can be formed of any suitable metal or metal alloy as is known in the art including, but not limited to, stainless steel, titanium and alloys thereof, cobalt and alloys thereof, chromium and alloys thereof, and so forth.

In some embodiments, the metal or metal alloy is a shape memory alloy.

In some embodiments, the metal or metal alloy is a nitinol (nickel-titanium) shape memory alloy.

FIG. 1B is a partial view of valve frame 10 similar to that shown in FIG. 1A in a reduced diameter state. This may result from a crimping process. As can be seen from FIGS. 1A and 1B, the length of the valve frame 10 increases when the diameter of the valve frame 10 is reduced as shown in FIG. 1B, and the diameter of the valve frame 10 increases when the length of the valve frame 10 is decreased.

In the embodiment shown in FIGS. 1A and 1B, the valve frame 10 is shown having an outer diameter (OD) of about 22 mm in its starting geometry shown in FIG. 1A and in the crimped state as shown in FIG. 1B, the OD is decreased to about 7.5 mm

FIG. 1C is a partial perspective view of a valve frame 10 similar to the one shown in FIGS. 1A and 1B in an expanded state.

FIGS. 1D and 1E are partial views of a single cell 14 illustrating the effect on a single cell 14 of a valve frame with an applied load simulating that which would be applied during a crimping process as shown in FIG. 1E. As can be seen, the diameter of the cell 14 decreases while the length increases.

FIGS. 1D and 1F illustrate the effect on a single cell 14 when the valve frame is compressed axially as during expansion as shown in FIG. 1F. The diameter of the cell 14 increases while the length of the cell 14 decreases.

FIGS. 2-4 are illustrative of various embodiments of a valve frame 10 having different cell geometries. Each of these embodiments is intended for illustrative purposes only, and not as a limitation on the scope of the invention. Other cell geometries may be employed without deviating from the scope of the present invention.

FIG. 2 is a partial view of an embodiment of a valve frame 10 wherein the plurality of cells 14 are diamond shaped. Each cell 14, as in FIGS. 1A-1C, is made up of struts 16 connected by peaks 18a and valleys 18b. Adjacent cells 14 are interconnected by connectors 20 and adjacent cylindrical segments 12 are also interconnected by connectors 20.

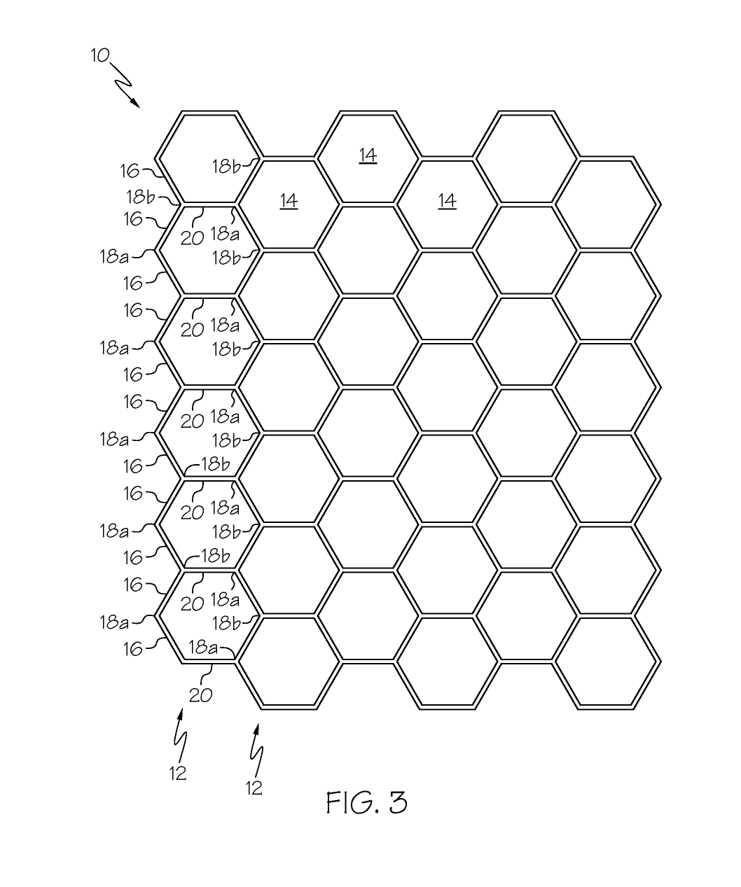

FIG. 3 is a partial view of an embodiment of a valve frame 10 wherein the plurality of cells 14 are honeycomb-shaped or hexagonal. Each cell 14, as in FIGS. 1A-2, is made up of a plurality of struts 16 connected by peaks 18a and valleys 18b. Adjacent cells 14 are interconnected by connectors 20 and adjacent cylindrical segments 12 are also interconnected by connectors 20.

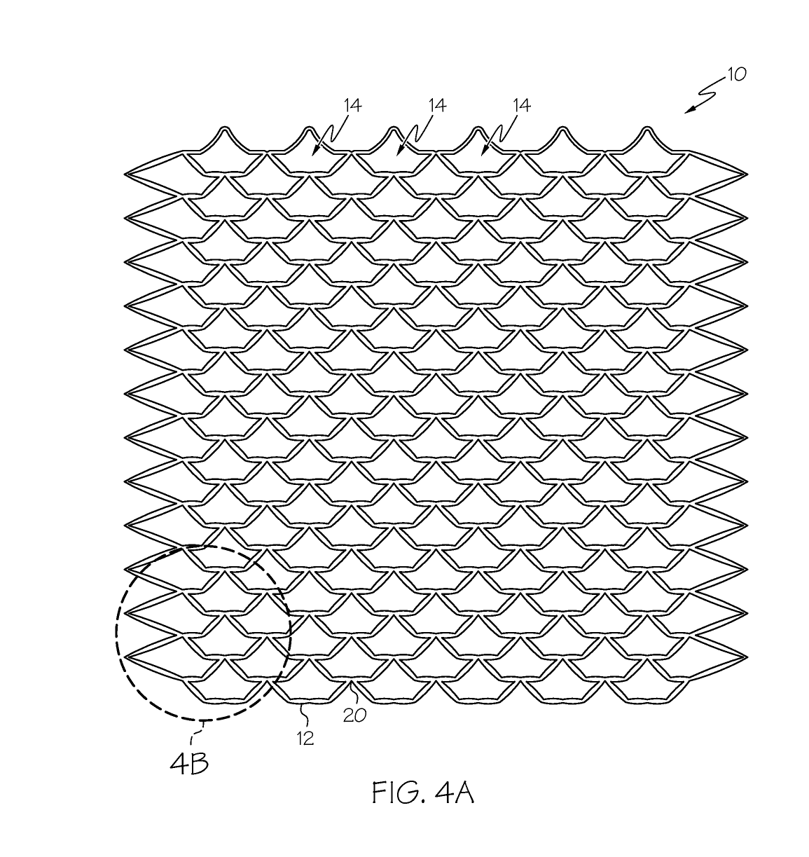

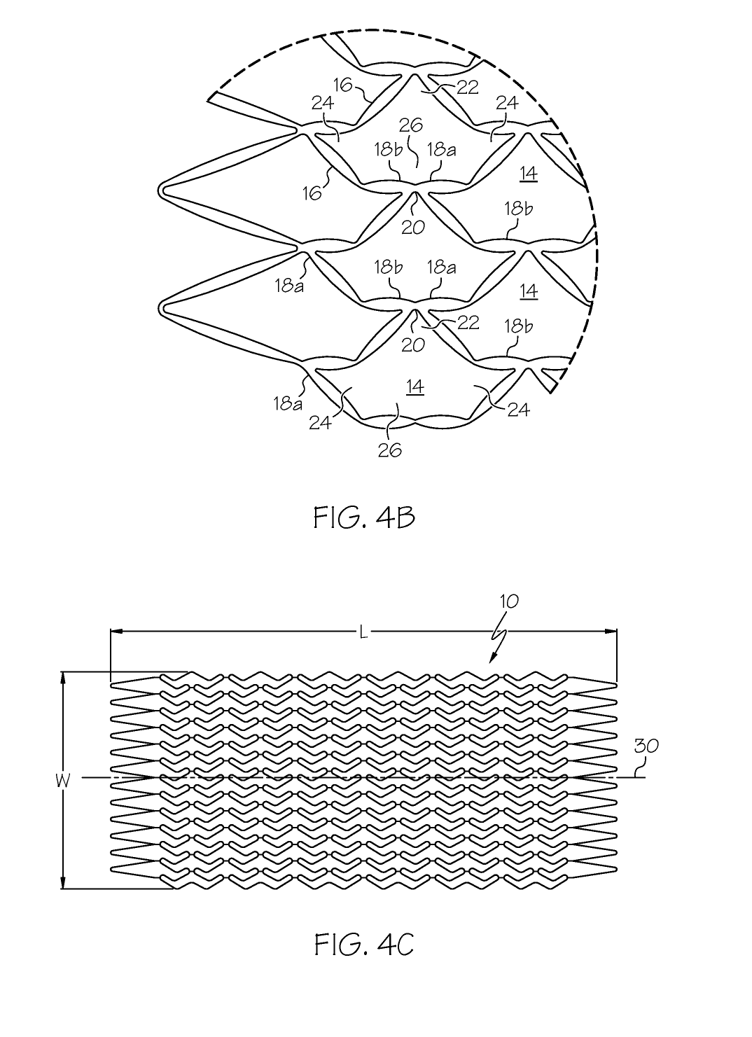

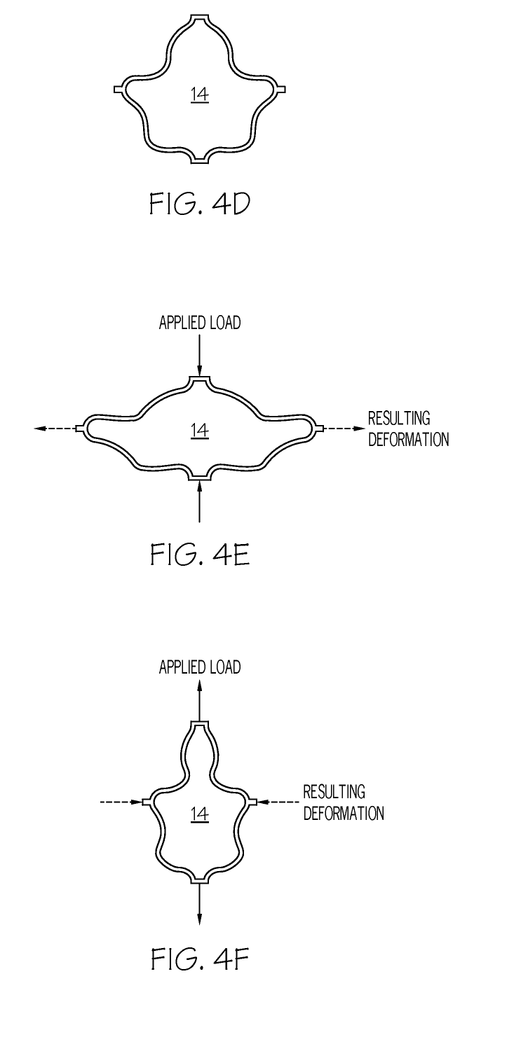

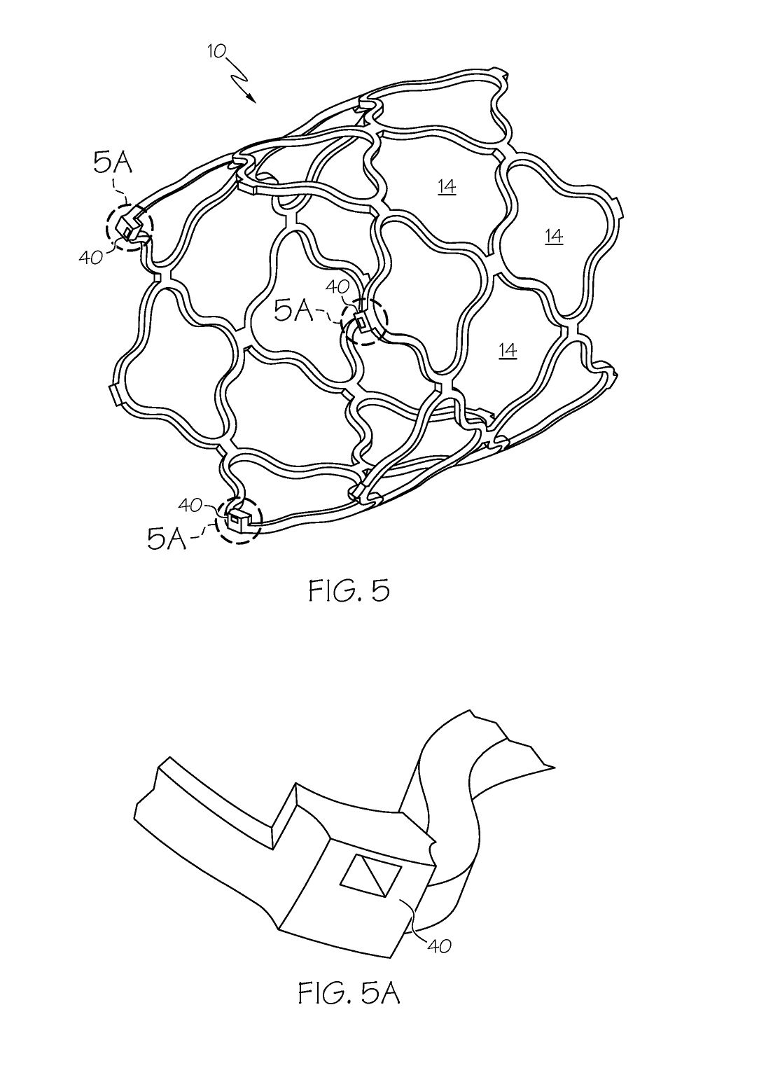

FIG. 4A is a partial view of an embodiment of a valve frame 10 wherein the plurality of cells 14 are bat-shaped cells. FIG. 4B is an enlarged portion taken at 4B in FIG. 4A for more clearly illustrating each cell shape. Each cell 14, as in FIGS. 1A-3, is made up of a plurality of struts 16 connected by peaks 18a and valleys 18b. Adjacent cells 14 are interconnected by connectors 20 and adjacent cylindrical segments 12 are also interconnected by connectors 20.

FIG. 4C illustrates a valve frame 10 similar to that shown in FIGS. 4A and 4B in a compressed state. Again, as in FIGS. 1A and 1B, the length of the valve frame 10 increases when the diameter of the valve frame 10 is reduced as shown in FIG. 4C, and the diameter of the valve frame 10 increases when the length of the valve frame 10 is decreased.

Alternatively, a similar pattern as that shown in FIG. 4A may be employed but with alternating cylindrical bands mirroring one another without the “bat-like” shape.

FIGS. 4D-4F illustrate the effect on a single cell 14 when an applied load simulating that of a crimping process is applied to the cell as shown in FIG. 4E, and when axial force is applied to the cell as during expansion of the valve frame as shown in FIG. 4F.

The relative lengths as shown in the drawings are not to scale, but are for illustrative purposes only.

While the above embodiments illustrate partial views of valve frames having a uniform pattern throughout the valve frame, some embodiments may include different patterns over the length of the frame. One example for alternating the pattern may be to achieve different valve frame diameters along the length of the valve when expanded.

Axial or circumferential sections may also differ in frequency of density of the circumferential segments.

In some embodiments, it is desirable to provide more flexibility in the proximal and/or distal end of the valve frame to facilitate positioning, deployment and better valve leaflet performance. The may be accomplished by decreasing the frequency of the density at the distal and/or proximal end of the valve frame.

The circumferential geometry of sections of the valve frame may be varied as well as material force characteristics.

Furthermore, while certain exemplary cell geometries have been disclosed herein, other cell geometries which achieve the same phenomenon during compression and expansion of the valve frame may be employed herein without departing from the scope of the present invention.

The valve frame 10, further suitably includes a locking mechanism 40 as shown in FIGS. 5 and 5A. The locking mechanism 40 can be used to reduce the length of the valve frame 10 thereby increasing the valve frame 10 OD or to lock the valve frame into place to prevent it from lengthening once placed. Once the desirable OD has been achieved, it can be suitably locked to fix the length of the valve frame thus preventing lengthening and inhibiting any change in frame diameter once the valve frame is positioned in a patient at the target site.

Any suitable locking mechanism may be employed provided that it is formed integrally with the valve frame.

Any of the valve frames disclosed herein may have valve leaflets coupled thereto. Heart valves may comprise one or more cuspids. In some embodiments, the valve is a bicuspid or tricuspid valve or has two or three valve leaflets.

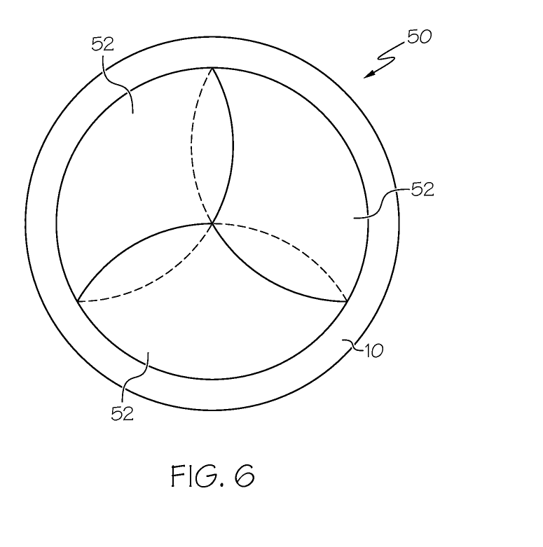

FIG. 6 illustrates generally at 50, a cross-sectional view of a valve frame 10 having three valve leaflets 52 coupled thereto.

The valve leaflets may be coupled or fixed to the valve frame using any method known in the art. In some embodiments, the valve may include posts known as valve supports and may include one post for each valve leaflet. Each post may include a slot wherein leaflet tissue is passed through the slot and sutured in place.

The device is deployed in a patient's valve opening having a diameter that is less than that of the valve frame's original diameter, and upon deployment, can rest against the patient's valve. In some embodiments wherein the valve is formed from a shape memory metal alloy, for example, it is possible that the valve can expand to a diameter greater than that of the patient's valve opening, can rest against the tissue, and then be locked into position to provide extra radial strength.

Alternatively, the valve upon deployment from the catheter or sheath, may be under deployed and does not rest against the tissue wall. By axially compressing the valve, however, and using the locking mechanism, the diameter of the valve can thus be increased to achieve proper positioning against the tissue wall.

Thus, the locking mechanism 40 can be used to reduce the length of the valve frame 10, resulting in an increase in frame diameter and once the position is satisfactory, the OD of the device is locked into place.

In the present device, the locking mechanism 40 is integral with the valve frame 10 and can be either laser cut into the valve frame or micro-machined into the valve frame 10.

In some embodiments, the locking mechanism 40 is a buckle or post.

Locking mechanisms of this type are disclosed in commonly assigned U.S. Pat. Nos. 7,329,279, 7,445,631, 7,748,389, 7,824,442, 7,959,666, 8,343,213 and 8,828,078, each of which is incorporated by reference herein in its entirety.

Providing a locking mechanism integral with the valve frame 10, rather than separately providing a locking mechanism and subsequently suturing it to the valve frame 10, can improve production efficiency and reduce variations from part to part variability that may occur during the manufacturing process.

The present device exhibits high radial strength making it more robust and resistant to deformation that may be imposed by heart muscles, while providing improved production efficiency over conventional braided valve frames. Moreover, the present device eliminates fretting wear that caused by relative motion of the strands in contact with one another as in the case with conventional braided valve frames.

Any of the embodiments of the valve frames illustrated herein can be used in both self-expanding as well as balloon expandable valves frames.

While specific examples of metal and metal alloys have been disclosed herein. Other materials can also be employed in forming the valve frame. Examples of materials employed in forming self-expanding frames include those formed from temperature-sensitive memory alloy which changes shape at a designated temperature or in a temperature range and include, but are not limited to, nitinol which is a nickel-titanium alloy, elgiloy which is a cobalt-chromium-nickel alloy, copper-aluminum-nickel, copper-zinc-aluminum and iron manganese-silicon alloys.

Other materials for use herein include, but are not limited to, medical grade stainless steel alloys (304, 316, 17-7 PH, 17-4 PH, etc.), titanium, tantalum, platinum, niobium, cobalt tungsten, molybdenum, titanium, and alloys and combinations thereof

Etching, heat-setting, chemical or electrochemical polishing and so forth, may be conducted on the valve frame to achieve medical implant-grade construction.

Polymer materials can also be employed herein including thermoplastic polymer materials and shape memory polymer materials.

The above lists are intended for illustrative purposes only, and not as a limitation on the scope of the present invention. Any suitable materials can be employed in forming the valve frame and such materials are well known in the art.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All published documents, including all US patent documents and US patent publications, mentioned anywhere in this application are hereby expressly incorporated herein by reference in their entirety. Any co-pending patent applications, mentioned anywhere in this application are also hereby expressly incorporated herein by reference in their entirety. Citation or discussion of a reference herein shall not be construed as an admission that such is prior art.

1. A transcatheter valve frame comprising:
a valve frame comprising an elongate tubular member having a longitudinal axis and a circumference, the elongate tubular member comprising a plurality of struts interconnected by peaks and valleys that define a closed cell construction comprising a plurality of interconnected cells, the valve frame is formed from a single piece of material comprising a metal or metal alloy, the valve frame having a diameter that increases upon axial compression; and
the valve frame comprising a locking mechanism integral with the valve frame configured to fix the diameter of the valve frame.

2. The transcatheter valve frame of claim 1 wherein the plurality of cells define a uniform pattern throughout the elongate tubular member.

3. The transcatheter valve frame of claim 1 wherein the plurality of cells define a uniform pattern throughout the elongated tubular member, the plurality of cells comprises arcuate-shaped diagonal elements.

4. The transcatheter valve frame of claim 3 comprising a plurality of cylindrical bands, within each cylindrical band, each of the arcuate-shaped diagonal elements is oriented in the same direction about the circumference of the elongate tubular member.

5. The transcatheter valve frame of claim 3 wherein the arcuate-shaped diagonal elements comprise a less arcuate shape in a stretched condition than in a non-stretched condition.

6. The transcatheter valve frame of claim 1 wherein the plurality of struts are connected by peaks and valleys are arranged in a plurality of cylindrical bands formed in the elongate tubular member, each cylindrical band forming a generally zig-zag pattern that forms a series of sequential diagonal elements connected to one another and extending about the circumference of the elongate tubular member.

7. The transcatheter valve frame of claim 6 comprising a plurality of longitudinal connectors extending between and connecting adjacent bands.

8. The transcatheter valve frame of claim 6 wherein each of the sequential diagonal element comprises first and second generally straight portions having first and second ends, the second ends being connected together by a curved portion, the first ends being connected to preceding and succeeding diagonal elements.

9. The transcatheter valve frame of claim 1 wherein the elongate tubular member is radially expandable between an unexpanded state and an expanded state.

10. The transcatheter valve frame of claim 1 wherein the plurality of cells are honeycomb-shaped.

11. The transcatheter valve frame of claim 1 wherein the plurality of cells are diamond-shaped.

12. The transcatheter valve frame of claim 1 wherein the plurality of cells are generally bat-shaped wherein each cell defines a head region, a tail region and opposing curved wing regions, and a plurality of connectors extending between and connecting adjacent cells.

13. The transcatheter valve frame of claim 12, the elongate tubular body comprising a plurality of cylindrical bands, each cylindrical band comprising a sequence of the generally bat-shaped cells, adjacent cylindrical bands are connected to one another by longitudinal connectors.

14. The transcatheter valve frame of claim 1 wherein the plurality of cells comprise a four-lobed shape wherein each lobe is uniformly spaced from an adjacent lobe.

15. The transcatheter valve frame of claim 1 wherein the locking mechanism is a buckle or post integral with the valve frame.

16. The transcatheter valve frame of claim 1 wherein the metal or metal alloy is a shape memory alloy.

17. The transcatheter valve frame of claim 1 wherein the metal of metal alloy is nitinol.

18. A method of making a valve frame, the method comprising:
providing a elongate tubular member formed from a continuous piece of material; and
cutting a pattern into the elongate tubular member, the pattern comprising a plurality of struts interconnected by peaks and valleys that define a closed cell construction comprising a plurality of interconnected cells to form the valve frame.

19. The method of claim 18 further comprising forming a locking mechanism in the valve frame.

20. The method of claim 19 comprising forming the locking mechanism in the valve frame by cutting or micromachining.
